(19) United States
(12) Patent Application Publication
(10) Pub. No.: US 2014/0230850 A1
(43) Pub. Date: Aug. 21, 2014

(54) RAMROD FOR MRI AND METHODS THEREOF

(71) Applicant: ASPECT IMAGING LTD., Shoham (IL)

(72) Inventor: Uri RAPOPORT, Moshav Ben Shemen (IL)

(21) Appl. No.: 13/906,833

(22) Filed: May 31, 2013

Related U.S. Application Data
(60) Provisional application No. 61/766,734, filed on Feb. 20, 2013.

(51) Int. Cl. A61B 19/00

(52) U.S. Cl. CPC A61B 19/34
USPC 134/8; 15/104.05

(57) ABSTRACT

A ramrod for cleaning the bore of an MRI device, including: a head with a cross-section substantially identical in shape to the MRI bore's cross-section, the cross section having size slightly smaller than the MRI's bore's cross-section to fit snugly and movably within the bore, the head having at least one end, the head having a longitudinal axis; and a rigid extended longitudinal body rigidly connected to the at least one end, the body having a longitudinal axis. The passage of the ramrod through the bore removes particulates from the bore.

### FIELD OF THE INVENTION

The present invention generally pertains to a system and method for cleaning the interior of MRI devices.

### BACKGROUND OF THE INVENTION

Since MRI devices include magnets producing high magnetic fields, they will, in use, attract metal towards them. Larger objects, such as stethoscopes, watches, or paperclips can routinely be kept away from the MRI, or removed by had if the accidentally enter the MRI bore. However, it is very difficult to keep debris such as metal fragments or dust from entering the MRI bore. Some examples of such fragments are broken bits from paper clips, fragments of broken needles, and tips from ballpoint pens. Once debris has entered the MRI bore, it will tend to stick to the pole magnets and will tend to remain within the bore of the MRI. Even for MRIs with main electromagnets, turning off the magnets will not clear the debris. It will to fall to the bottom of the bore, but will not be removed from the bore and will reattach to the magnets when the MRI is next turned on.

This debris, if allowed to build up, can interfere with both the uniformity and the strength of the magnetic fields, reducing the quality of the images generated by the MRI.

U.S. Pat. No. 5,918,342 discloses a device to sanitize the bore of a medical MRI by applying disinfectant substances to the bore, the device comprising two sponge elements, one with arcuate surface, for cleaning curved parts of the bore and one with planar surface for cleaning flat parts of the bore.

However, U.S. Pat. No. 5,918,342 discloses a sanitizing device, intended to disinfect the bore. The sanitizing device can be used for removing soluble material from the bore; the patent discloses use of the sanitizing device to remove urine or feces from the bore. However, U.S. Pat. No. 5,918,342 does not disclose a device intended to remove solids from the bore, and, more particularly, it does not disclose a device for removal of debris, especially metallic debris, from the bore.

It is therefore a long felt need to provide a method of cleaning insoluble solid material from the bore of an MRI.

### SUMMARY OF THE INVENTION

It is an object of the present invention to disclose a ramrod for cleaning insoluble solid material from the bore of an MRI.

It is another object of the present invention to disclose a ramrod for cleaning the bore of an MRI device. The ramrod comprises a substantially cylindrical head with cross-section substantially identical in shape to said MRI bore's cross-section, said cross section having size slightly smaller than said MRI's bore's cross-section to fit snugly and movably within said bore, said head having at least one end, said head having a longitudinal axis; and a rigid extended longitudinal body rigidly connected to said at least one end, said body having a longitudinal axis; wherein passage of said ramrod through said bore removes particulates from said bore.

It is in the scope of the invention wherein the longitudinal body is rigid, flexible or both, in a manner that at least one first portion is rigid and at least one second neighboring portion is flexible.

It is another object of the present invention to disclose the ramrod described above, wherein said head has shape selected from a group consisting of substantially cylindrical, substantially spherical, substantially ovoid, substantially ellipsoidal, substantially conical, comprising one or more frustums, and any combination thereof.

It is another object of the present invention to disclose the ramrod described above, wherein the material of said ramrod is selected from a group consisting of rigid plastic, hard rubbery plastic, wood, carbon fiber, a non-metallic laminate, and any combination thereof.

It is another object of the present invention to disclose the ramrod described above, wherein the material of the surface of said ramrod is selected from a group consisting of rigid plastic, rubbery plastic, foam, woven material, non-woven material, chamois cloth, carbon fiber and any combination thereof.

It is another object of the present invention to disclose the ramrod described above, wherein at least a portion of said ramrod is hollow.

It is another object of the present invention to disclose the ramrod described above, wherein the angle subtended between said longitudinal axis of said head and said longitudinal axis of said body is between 0 and about 5°.

It is another object of the present invention to disclose the ramrod described above, wherein said extended longitudinal body has cross-section selected from circular, oval, elliptical, polygonal, stellate, and any combination thereof.

It is another object of the present invention to disclose the ramrod described above, wherein at least one pair of frustums is joined at their wide ends, at their narrow ends, narrow end to wide end, and any combination thereof.

It is another object of the present invention to disclose the ramrod described above, wherein at least one pair of frustums are separated by a substantially cylindrical section.

It is another object of the present invention to disclose the ramrod described above, wherein said head comprises at least one groove, said groove substantially parallel to said longitudinal axis, substantially perpendicular to said longitudinal axis, said groove disposed spirally about said head, and any combination thereof.

It is another object of the present invention to disclose the ramrod described above, wherein said at least one groove is less than about 5 mm deep at their deepest point.

It is another object of the present invention to disclose the ramrod described above, wherein said at least one groove has a profile that is at least one of V-shaped, U-shaped and any combination thereof.

It is another object of the present invention to disclose the ramrod described above, wherein said extended longitudinal body comprises at least one hand grip.

It is another object of the present invention to disclose the ramrod described above, wherein said at least one hand grip comprises at least one portion selected from a group consisting of finger grooves, a pistol grip, and any combination thereof.

It is another object of the present invention to disclose the ramrod described above, wherein said at least one hand grip is comprised of a member of a group consisting of rigid plastic, rubbery plastic, foam, woven material, non-woven material, chamois cloth, wood, carbon fiber and any combination thereof.

It is another object of the present invention to disclose the ramrod described above, additionally comprising at least one brush attached to at least some portion of its external surface, said brush adapted to contact at least some portion of the wall of the bore of said MRI.

It is another object of the present invention to disclose the ramrod described above, wherein said brush is attached to said ramrod in a manner selected from rigidly or flexibly.

It is another object of the present invention to disclose the ramrod described above, wherein said brush is disposed about said ramrod in a manner selected from a group consisting of at least one sector of a substantially circular ring around said ramrod, at least one sector of a substantially elliptical ring around said ramrod, at least one sector of a substantially oval ring around said ramrod, at least one sector of a hyperbola around said ramrod, at least one sector of a spiral around said ramrod, a line substantially parallel to the longitudinal axis of said ramrod, a substantially circular patch on said ramrod, a substantially elliptical patch on said ramrod, a substantially oval patch on said ramrod, a substantially polygonal patch on said ramrod, a spiral around said ramrod, a patch substantially parallel to the longitudinal axis of said ramrod, randomly, and any combination thereof.

It is another object of the present invention to disclose the ramrod described above, wherein said at least one brush is in one of the following positions: at the distal end of the head, at the proximal end of the head, on the body at a predetermined first distance form the proximal end of the head, on the head at a predetermined second distance from the distal end of the head and any combination thereof.

It is another object of the present invention to disclose the ramrod described above, wherein said first distance is in the range of 0 to approximately 50% of the length of the body.

It is another object of the present invention to disclose the ramrod described above, additionally comprising a cover removably mountable on said head.

It is another object of the present invention to disclose the ramrod described above, wherein said cover is disposable.

It is another object of the present invention to disclose the ramrod described above, wherein said cover fits said head snugly such that said cover moves substantially with said head during cleaning.

It is another object of the present invention to disclose the ramrod described above, wherein said cover is comprised of flexible plastic, rubbery plastic, foam, woven material, non-woven material, chamois cloth, and any combination thereof.

It is another object of the present invention to disclose the ramrod described above, wherein said cover is comprised of one or more layers.

It is another object of the present invention to disclose the ramrod described above, additionally comprising at least one brush on at least some portion of its external surface, said at least one brush adapted to contact the surface of said bore.

It is another object of the present invention to disclose the ramrod described above, wherein said brush is attached to said cover in a manner selected from rigidly or flexibly.

It is another object of the present invention to disclose the ramrod described above, wherein said at least one brush is in one of the following positions: at the distal end of the head, at the proximal end of the head, on the head at a predetermined second distance from the distal end of the head and any combination thereof.

It is another object of the present invention to disclose the ramrod described above, wherein said second distance is in the range of 0 to approximately 100% of the length of the head.

It is another object of the present invention to disclose the ramrod described above, wherein said brush is disposed about said cover in a manner selected from a group consisting of at least one sector of a substantially circular ring around said ramrod, at least one sector of a substantially elliptical ring around said ramrod, at least one sector of a substantially oval ring around said ramrod, at least one sector of a hyperbola around said ramrod, at least one sector of a spiral around said ramrod, a line substantially parallel to the longitudinal axis of said ramrod, a substantially circular patch on said ramrod, a substantially elliptical patch on said ramrod, a substantially oval patch on said ramrod, a substantially polygonal patch on said ramrod, a spiral around said ramrod, a patch substantially parallel to the longitudinal axis of said ramrod, randomly, and any combination thereof.

It is another object of the present invention to disclose the ramrod described above, wherein said brush is comprised of at least one wire, at least one continuous portion and any combination thereof.

It is another object of the present invention to disclose the ramrod described above, wherein said brush is comprised of rigid plastic, rubbery plastic, foam, carbon fiber, and any combination thereof.

It is another object of the present invention to disclose the ramrod described above I, wherein said at least one sector subtends an angle of from 0 to 360°.

It is another object of the present invention to disclose the ramrod described above, wherein the perimeter of said patch has a shape selected from a group consisting of substantially circular, substantially elliptical, substantially oval, substantially polygonal, substantially stellate, irregular, and any combination thereof.

It is another object of the present invention to disclose the ramrod described above, wherein said cover comprises a top shaped to fit snugly to at least some portion of the distal end of said ramrod head.

It is another object of the present invention to disclose the ramrod described above, wherein said cover comprises a bottom shaped to fit snugly to at least some portion of the proximal end of said ramrod head.

It is another object of the present invention to disclose a standard of care protocol for cleaning the bore of an MRI, comprising providing a ramrod with a substantially cylindrical head with cross-section substantially identical in shape to said MRI bore's cross-section, said cross section having size slightly smaller than said MRI's bore's cross-section to fit snugly and movably within said bore, said head having at least one end, said head having a longitudinal axis; and a rigid extended longitudinal body rigidly connected to said at least one end, said body having a longitudinal axis; specifying at least one cleaning criterion; waiting until at least one said at least one cleaning criterion is fulfilled; and reciprocating said ramrod through said bore, thereby maintaining the cleanliness of said bore; wherein by reciprocating said ramrod through said bore particulates from said bore are removed.

It is another object of the present invention to disclose the standard of care protocol, wherein said cleaning criterion is selected from a group consisting of: before use of said MRI and after a predefined number of uses of said MRI; after use of said MRI, said use being the last of a predefined number of uses of said MRI; after a predefined time interval has elapsed; on a predefined day; on a predefined date; a predefined quantity of particulates are exceeded within the bore; and any combination thereof.

It is another object of the present invention to disclose the standard of care protocol, wherein said predefined number of uses of said MRI is greater than or equal to one.

It is another object of the present invention to disclose the standard of care protocol, comprising additional steps of examining said bore and determining the quantity of particulates therein.

It is another object of the present invention to disclose the standard of care protocol, wherein said examination of said bore is conducted visually, mechanically, optically and any combination thereof.

It is another object of the present invention to disclose a method of cleaning the bore of an MRI device, comprising providing a ramrod with a substantially cylindrical head with cross-section substantially identical in shape to said MRI bore's cross-section, said cross section having size slightly smaller than said MRI's bore's cross-section to fit snugly and movably within said bore, said head having at least one end, said head having a longitudinal axis; and a rigid extended longitudinal body rigidly connected to said at least one end, said body having a longitudinal axis; and reciprocating said ramrod through said bore; wherein by reciprocating said ramrod through said bore particulates from said bore are removed.

It is another object of the present invention to disclose the method of cleaning the bore, additionally comprising a step of selecting said head shape from a group consisting of substantially cylindrical, substantially spherical, substantially ovoid, substantially ellipsoidal, substantially conical, comprising one or more frustums, and any combination thereof.

It is another object of the present invention to disclose the method of cleaning the bore, additionally comprising a step of providing said longitudinal body to be rigid, flexible or both, in a manner that at least one first portion is rigid and at least one second neighboring portion is flexible.

It is another object of the present invention to disclose the method of cleaning the bore, additionally comprising a step of selecting the material of said ramrod from a group consisting of rigid plastic, hard rubbery plastic, wood, carbon fiber, a non-metallic laminate, and any combination thereof.

It is another object of the present invention to disclose the method of cleaning the bore, additionally comprising a step of selecting the material of the surface of said ramrod from a group consisting of rigid plastic, rubbery plastic, foam, woven material, non-woven material, chamois cloth, carbon fiber and any combination thereof.

It is another object of the present invention to disclose the method of cleaning the bore, additionally comprising a step of providing said ramrod such that at least a portion of said ramrod is hollow.

It is another object of the present invention to disclose the method of cleaning the bore, additionally comprising a step of setting the angle subtended between said longitudinal axis of said head and said longitudinal axis of said body between 0 and about 5°.

It is another object of the present invention to disclose the method of cleaning the bore, additionally comprising a step of selecting the cross-section of said extended longitudinal body from a group consisting of circular, oval, elliptical, polygonal, stellate, and any combination thereof.

It is another object of the present invention to disclose the method of cleaning the bore, additionally comprising a step of comprising said head of at least one pair of frustums, said frustums joined at their wide ends, at their narrow ends, narrow end to wide end, and any combination thereof.

It is another object of the present invention to disclose the method of cleaning the bore, additionally comprising a step of separating at least one pair of frustums by a substantially cylindrical section.

It is another object of the present invention to disclose the method of cleaning the bore, additionally comprising a step of providing said head comprising at least one groove, said groove substantially parallel to said longitudinal axis, substantially perpendicular to said longitudinal axis, said groove disposed spirally about said head, and any combination thereof.

It is another object of the present invention to disclose the method of cleaning the bore, additionally comprising a step of selecting the depth of said at least one groove to be less than about 5 mm deep at their deepest point.

It is another object of the present invention to disclose the method of cleaning the bore, additionally comprising a step of selecting the profile of said at least one groove to be V-shaped, U-shaped and any combination thereof.

It is another object of the present invention to disclose the method of cleaning the bore, additionally comprising a step of providing said extended longitudinal body comprising at least one hand grip.

It is another object of the present invention to disclose the method of cleaning the bore, additionally comprising a step of providing said at least one hand grip comprising at least one portion selected from a group consisting of finger grooves, a pistol grip, and any combination thereof.

It is another object of the present invention to disclose the method of cleaning the bore, additionally comprising a step of comprising said at least one hand grip from a member of a group consisting of rigid plastic, rubbery plastic, foam, woven material, non-woven material, chamois cloth, wood, carbon fiber and any combination thereof.

It is another object of the present invention to disclose the method of cleaning the bore, additionally comprising a step of providing at least one brush attached to at least some portion of said ramrod's external surface, said brush adapted to contact at least some portion of the wall of the bore of said MRI.

It is another object of the present invention to disclose the method of cleaning the bore, additionally comprising a step of attaching said brush to said ramrod in a manner selected from rigidly or flexibly.

It is another object of the present invention to disclose the method of cleaning the bore, additionally comprising a step of disposing said brush about said ramrod in a manner selected from a group consisting of at least one sector of a substantially circular ring around said ramrod, at least one sector of a substantially elliptical ring around said ramrod, at least one sector of a substantially oval ring around said ramrod, at least one sector of a hyperbola around said ramrod, at least one sector of a spiral around said ramrod, a line substantially parallel to the longitudinal axis of said ramrod, a substantially circular patch on said ramrod, a substantially elliptical patch on said ramrod, a substantially oval patch on said ramrod, a substantially polygonal patch on said ramrod, a spiral around said ramrod, a patch substantially parallel to the longitudinal axis of said ramrod, randomly, and any combination thereof.

It is another object of the present invention to disclose the method of cleaning the bore, additionally comprising a step of positioning said at least one brush in one of the following positions: at the distal end of the head, at the proximal end of the head, on the body at a predetermined first distance form the proximal end of the head, on the head at a predetermined second distance from the distal end of the head and any combination thereof.

It is another object of the present invention to disclose the method of cleaning the bore, additionally comprising a step of selecting said first distance to be in the range of 0 to approximately 50% of the length of the body.

It is another object of the present invention to disclose the method of cleaning the bore, additionally comprising a step of providing a cover removably mountable on said head.

It is another object of the present invention to disclose the method of cleaning the bore, additionally comprising a step of selecting said cover to be disposable.

It is another object of the present invention to disclose the method of cleaning the bore, additionally comprising a step of fitting said cover snugly to said head such that said cover moves substantially with said head during cleaning.

It is another object of the present invention to disclose the method of cleaning the bore, additionally comprising a step of comprising said cover of flexible plastic, rubbery plastic, foam, woven material, non-woven material, chamois cloth, and any combination thereof.

It is another object of the present invention to disclose the method of cleaning the bore, additionally comprising a step of comprising said cover of one or more layers.

It is another object of the present invention to disclose the method of cleaning the bore, additionally comprising a step of providing at least one brush on at least some portion of said cover's external surface, said at least one brush adapted to contact the surface of said bore.

It is another object of the present invention to disclose the method of cleaning the bore, additionally comprising a step of attaching said brush to said cover in a manner selected from rigidly or flexibly.

It is another object of the present invention to disclose the method of cleaning the bore, additionally comprising a step of positioning said at least one brush in one of the following positions: at the distal end of the head, at the proximal end of the head, on the head at a predetermined second distance from the distal end of the head and any combination thereof.

It is another object of the present invention to disclose the method of cleaning the bore, additionally comprising a step of selecting said second distance to be in the range of 0 to approximately 100% of the length of the head.

It is another object of the present invention to disclose the method of cleaning the bore, additionally comprising a step of disposing said brush about said cover in a manner selected from a group consisting of at least one sector of a substantially circular ring around said ramrod, at least one sector of a substantially elliptical ring around said ramrod, at least one sector of a substantially oval ring around said ramrod, at least one sector of a hyperbola around said ramrod, at least one sector of a spiral around said ramrod, a line substantially parallel to the longitudinal axis of said ramrod, a substantially circular patch on said ramrod, a substantially elliptical patch on said ramrod, a substantially oval patch on said ramrod, a substantially polygonal patch on said ramrod, a spiral around said ramrod, a patch substantially parallel to the longitudinal axis of said ramrod, randomly, and any combination thereof.

It is another object of the present invention to disclose the method of cleaning the bore, additionally comprising a step of providing said brush to be made of at least one wire, at least one continuous portion and any combination thereof.

It is another object of the present invention to disclose the method of cleaning the bore, additionally comprising a step of providing said brush to be made of rigid plastic, rubbery plastic, foam, carbon fiber, and any combination thereof.

It is another object of the present invention to disclose the method of cleaning the bore, additionally comprising a step of selecting the angle subtended by said at least one sector to be from 0 to 360°.

It is another object of the present invention to disclose the method of cleaning the bore, additionally comprising a step of selecting the shape of the perimeter of said patch from a group consisting of substantially circular, substantially elliptical, substantially oval, substantially polygonal, substantially stellate, irregular, and any combination thereof.

It is another object of the present invention to disclose the method of cleaning the bore, additionally comprising a step of comprising said cover with a top shaped to fit snugly to at least some portion of the distal end of said ramrod head.

It is another object of the present invention to disclose the method of cleaning the bore, additionally comprising a step of comprising said cover with a bottom shaped to fit snugly to at least some portion of the proximal end of said ramrod head.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 depicts the prior art;

FIG. 2a-d schematically illustrate ramrods according a few embodiments of the present invention;

FIG. 3 schematically illustrates a straight handgrip for the ramrod;

FIG. 4 schematically illustrates a pistol grip for the ramrod;

FIG. 5 schematically illustrates a double grip for the ramrod;

FIG. 6a-d schematically illustrates a ramrod with brushes; and

FIG. 7 schematically illustrates a ramrod with a cover.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a ramrod for cleaning insoluble solid material from the bore of an MRI.

The term ‘approximately’ hereinafter refers to a value being within ±25% of the defined measure.

The term ‘substantially’ hereinafter refers to a value being within ±10% of the defined measure.

The term ‘slightly’ hereinafter refers to a value being within ±5% of the defined measure.

The term ‘plurality’ hereinafter refers to any number greater than one.

The term ‘cylinder’ or ‘cylindrical’ hereinafter refers to a surface generated by a straight line intersecting and moving along a closed plane curve while remaining perpendicular to the closed plane curve. As used herein, a cylindrical solid is bounded at each end by a perimeter of the shape of the closed plane curve, with the ends being either substantially flat or substantially conical.

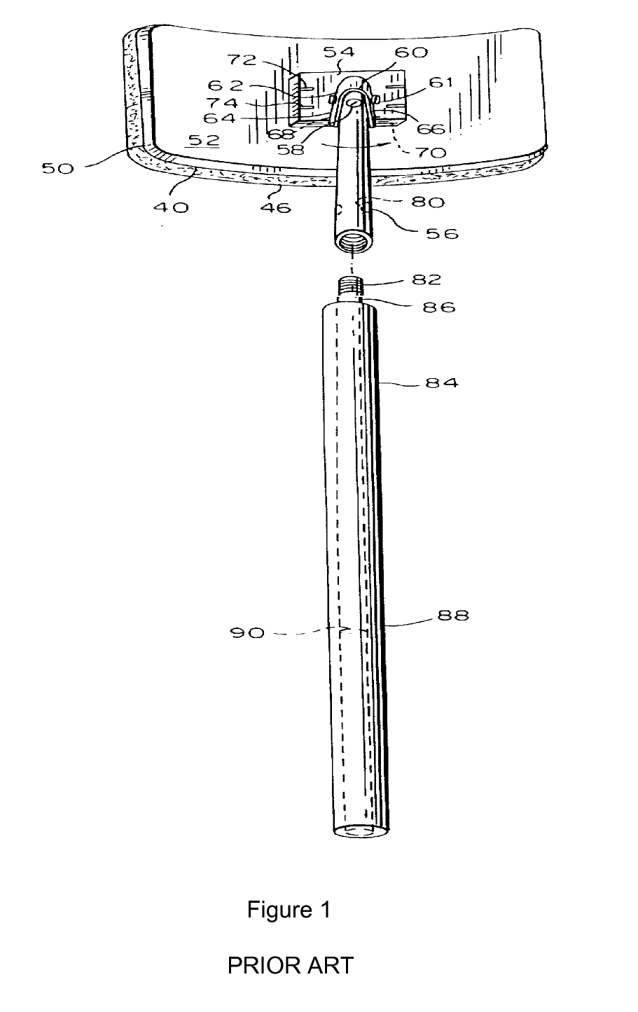

The prior art discloses a sanitizing device adapted to apply disinfectant to the bore of an MRI. An example of the prior art is shown in FIG. 1. The sanitizing device comprises a cleaning element 40 from a cleaning head 50 attached to a mounting plate 52 which is arcuate and is a segment of a cylinder having a radius R. Projecting from the mounting plate 52 is a boss 54. The boss 54 has a socket 56 pivoted thereto by a pivot pin 58, which pivot pin 58 extends perpendicularly from, or in a radial direction from, the plate 52. The pin 58 restrains the socket 56 to rotation about an axis 60 which extends perpendicular to the void of FIG. 1 but prevents the socket from rotating toward the axis 60. In other words, the socket 56 always extends substantially perpendicular to the axis 60.

Pivotally mounted on the socket 56 by a bolt 61 is a latch 62 which has two detent arms 64 and 66 which are received in the first slots 68 and 70 when the socket 56 is in the orientation shown in FIG. 1 and in second slots 72 and 74 when the socket 56 is rotated to a second position in which the socket extends in a direction perpendicular to the direction shown in FIG. 1.

The socket 56 has a threaded nut 80 therein which receives the threaded end 82 of a telescoping handle 84. The telescoping handle 84 includes an inner shaft 86 which has the threads 82 thereon and an outer tube 88 which receives the inner shaft. The inner shaft 86 has an eccentric 90 thereon, so that when the tube 88 is rotated with respect to the shaft 86, the tube and shaft lock with respect to one another. In this way, the length of the handle telescoping 84 is adjusted and locked. As will be further explained hereinafter, it is necessary to be able to rapidly detach the handle 84 from the first head 40 so that a second cleaning head adding the cleaning element 42 thereon may be substituted for the first head.

The prior art device is intended to disinfect the MRI bore, and can also be used to remove soluble matter such as urine or feces from the MRI bore. However, it is not designed to remove non-soluble matter from the MRI bore and will have difficulty doing so, as the rotatable head will tend to rotate around adhering solid matter rather than removing it. Furthermore, the prior art device is intended to clean the interior of large MRIs, large enough to contain entire human bodies.

The present device, a ramrod, is designed to remove solids, especially metallic debris, from the bore of an MRI, particularly smaller MRIs intended for imaging small animals such as rats or mice or for use in industrial applications such as inspection or quality control.

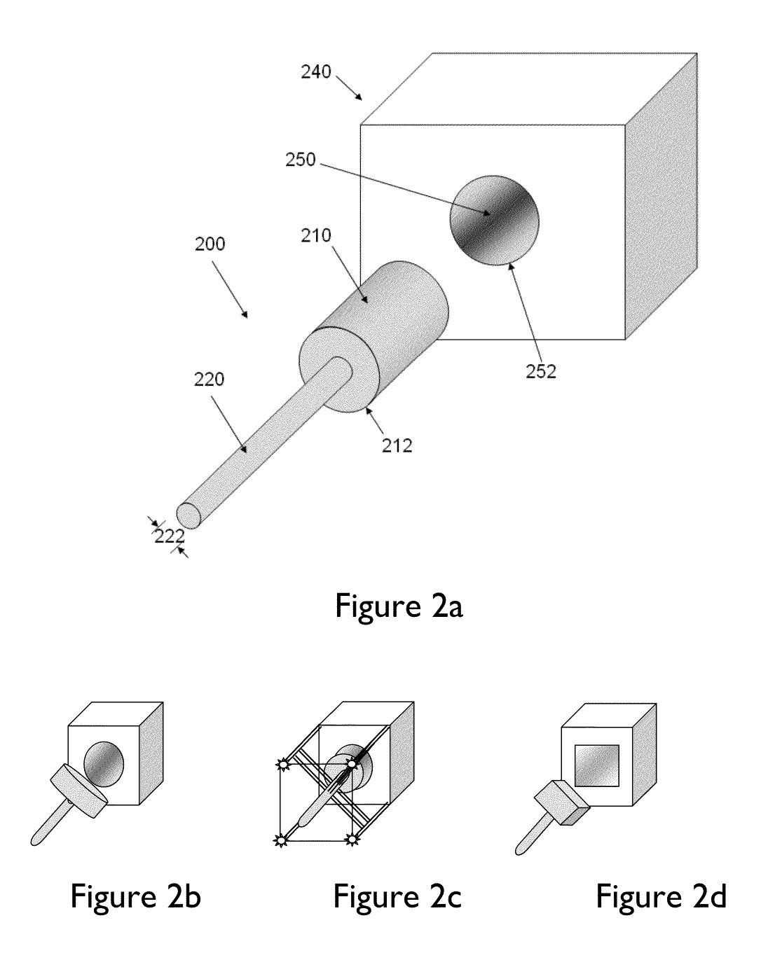

In reference to FIG. 2a, an embodiment is shown of the device disclosed herein, which comprises a ramrod 200 with a substantially cylindrical head 210 of cross-sectional shape 212 substantially identical to the shape 252 of the bore 250 of MRI 240 and cross-sectional area slightly smaller than the bore 250, the head 210 fitting snugly and movably within the bore. The ramrod has an extended longitudinal body 220, substantially cylindrical in shape, rigidly attached to one end of the head 210.

In some embodiments, the longitudinal axis of the extended longitudinal body is collinear with the longitudinal axis of the head. In other embodiments, it is parallel to the longitudinal axis of the head. In yet other embodiments, it is set at an angle to the longitudinal axis of the head. This angle can be up to approximately 5°.

In some embodiments, the extended longitudinal body is wholly rigid. In other embodiments, it comprises at least one rigid portion and at least one flexible portion. In some of these embodiments, the flexible portion enables portions of the bore to be cleaned more easily.

In reference to FIGS. 2b and 2d, embodiments are shown of devices for an MRI having a bore with circular cross section and polygonal cross section, respectively, disclosed herein, wherein FIG. 2b discloses a ramrod with a circular cross section, FIG. 2c discloses a ramrod with spherical head and FIG. 2d discloses a ramrod with a polygonal cross-section.

In reference to FIG. 2c, a mechanical ramrod reciprocating mechanism is disclosed. This mechanical, hydraulic and/or electrical mechanism facilitates an easy, effective and safe passage of the ramrod through the bore of the MRI.

In preferred embodiments, the longitudinal axis of the head 210 is collinear with the longitudinal axis of the body 220. In other embodiments, the longitudinal axis of the head 210 is at angle of up to 5° to the longitudinal axis of the body 220.

In some embodiments, the head 210 of the ramrod 200 is substantially cylindrical. In other embodiments, the head 210 forms a frustum. The wide end of the frustum can be at the distal end of the head 210, or it can be at the proximal end of the head 210. In yet other embodiments, the head 210 can comprise two frustums, either with the wide ends of the frustums touching each other, or with the narrow ends of the frustums touching each other. In yet other embodiments, the frustums are separated by a cylindrical section. In yet other embodiments, a plurality of frustums are used, so that the side of the head 210 forms a series of ridges; if the head is observed “side-on”, the longitudinal edges of the head will appear to be serrated.

In other embodiments, the head comprises a series of grooves adapted to contain the particulate matter after its removal from the bore of the MRI. These grooves can be substantially parallel to the longitudinal axis of the head, substantially perpendicular to the longitudinal axis of the head, or can be disposed spirally about the head. Perpendicular grooves can form complete rings about the head or can form sectors of a ring. Similarly, longitudinal or spiral grooves can extend the entire length of the head or only partway along or around the head. Preferably, the grooves are shallow, preferably less than about 5 mm deep at their deepest point. The grooves can be V-shaped, U-shaped, or any combination thereof.

In other embodiments, the head forms a sphere, ellipsoid or ovoid. Said spherical, elliptical or ovoid head can comprise grooves adapted to contain the particulate matter after its removal from the bore of the MRI. These grooves can be substantially parallel to the longitudinal axis of the ramrod, substantially perpendicular to the longitudinal axis of the ramrod, or can be disposed spirally about the head. Perpendicular grooves can form complete rings about the head or can form sectors of a ring. Similarly, longitudinal or spiral grooves can extend the entire length of the head or only partway along or around the head. Preferably, the grooves are shallow, preferably less than about 5 mm deep at their deepest point. The grooves can be V-shaped, U-shaped, or any combination thereof.

Preferably, the body 220 has a diameter 222 smaller than the diameter of the head 210.

In preferred embodiments, the cross-section of the body 220 is either substantially circular or substantially polygonal. However, the cross-section can be stellate or have any desired shape.

The ramrod is preferably made of non-metallic material. It can be made substantially of any rigid plastic known in the art, of a stiff rubbery plastic, of wood, of carbon fiber, of a non-metallic laminate, or of any combination thereof. The head 210, the body 220 or both can be hollow. The surface of the head 210, the body 220 or both can be comprised of rigid plastic, rubbery plastic, foam, woven material, non-woven material, chamois cloth, carbon fiber or any combination thereof.

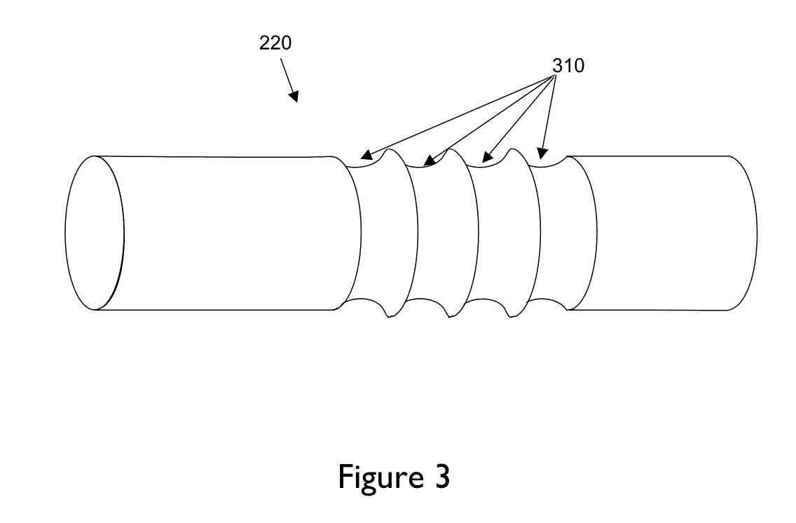

In reference to FIG. 3, in some embodiments, the body 220 comprises at least one hand grip 300. In some variants of these embodiments, the hand grip comprises a series of finger grooves 310 to improve the ability of a user to grip the ramrod firmly. The grip 300 can comprise a soft or compressible cover or the grip 300 can be comprised of a soft or compressible material, in order to make it more comfortable for a user to grasp the ramrod firmly. The at least one grip 300 can be comprised of rigid plastic, rubbery plastic, foam, woven material, non-woven material, chamois cloth, wood, carbon fiber or any combination thereof.

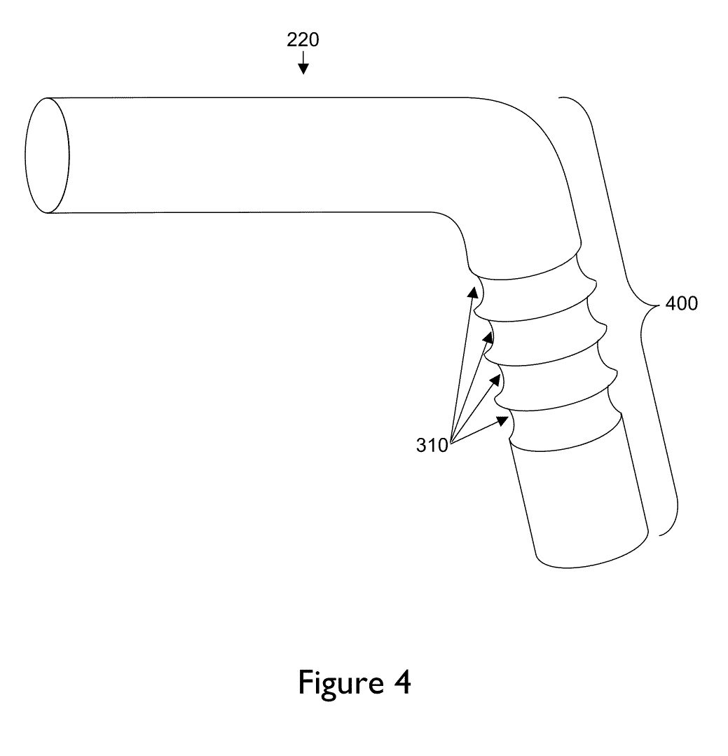

In reference to FIG. 4, in other variants of embodiments with a hand grip the body 220 comprises a pistol grip 400. As shown in FIG. 4, the pistol grip 400 can further comprise a series of finger grooves 310 to improve the ability of a user to grip the ramrod firmly.

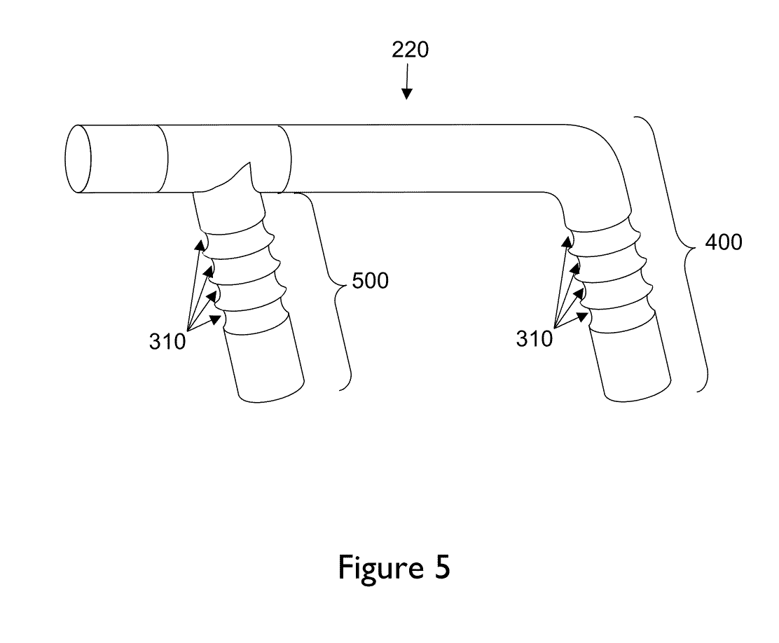

In reference to FIG. 5, in other variants of embodiments with a hand grip, the body 220 comprises two hand grips. In some variants of embodiments with two hand grips, as shown in FIG. 5, the rear grip comprises a pistol grip 400 and the fore grip also comprises a pistol grip 500. In other variants, one or both of the grips comprise straight grips. Either or both grips can comprise finger grooves 310.

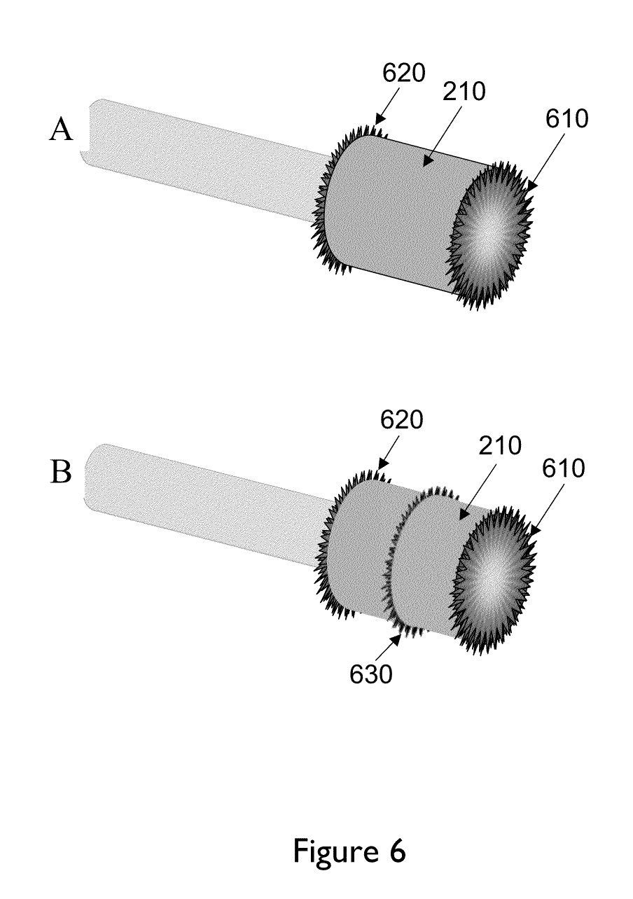

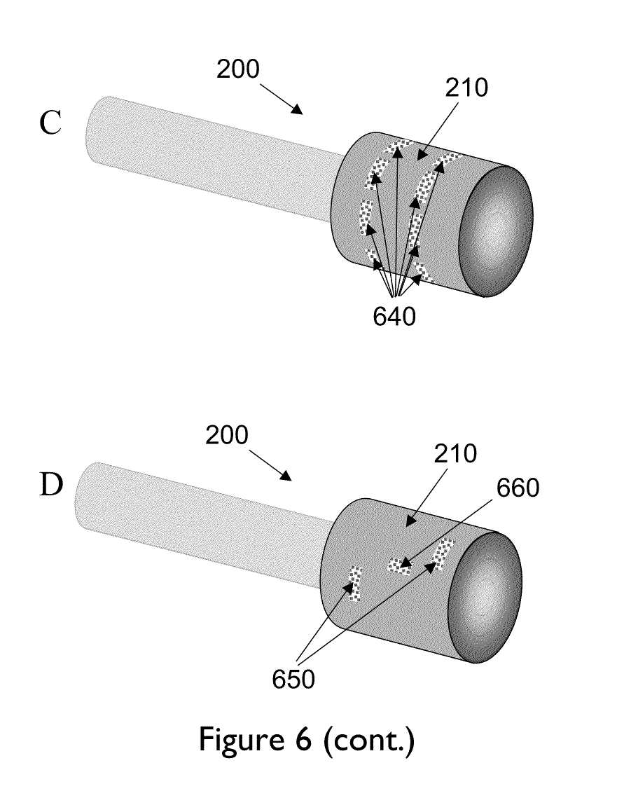

In reference to FIG. 6, in some embodiments, the ramrod comprises at least one brush (610, 620, 630, 640, 650, 660) firmly attached to at least one portion of the ramrod and with at least one portion of the brush enabled to contact at least some portion of the wall of the bore, the brush adapted to remove particulates from the walls of the bore.

The brush is preferably not of metal. The brush can be comprised of rigid plastic, rubbery plastic, foam, carbon fiber, and any combination thereof. The brush can comprise wire, continuous portions, and any combination thereof.

The brush is preferably removably attached to the ramrod so that it can be replaced if worn or dirty, although it can be permanently attached. The brush is preferably rigidly attached to the ramrod, although it can be flexibly attached thereunto.

In FIG. 6A, the ramrod is shown with two sets of brushes, one (610) at the distal end of the head 210, the other (620) at the proximal end of the head 210.

Brushes can be attached to the head 210 of the ramrod or attached to the body 220 of the ramrod. Brushes attached to the body 220 of the ramrod are preferably attached to the distal half of the ramrod, from the proximal end of the head 210 to the center of the ramrod body, which can also be described as in the range of 0 to approximately 50% of the length of the body, as measured from the proximal end of the head 210.

One or more intercalary brushes can be attached to the head 210; intercalary brushes can be attached anywhere on the head 210, at a distance from the proximal end of the head of 0 to approximately 100% of the length of the head. FIG. 6B shows a non-limiting example of a ramrod with intercalary brushes. In FIG. 6B, one intercalary brush (630) is shown, encircling the head 210 about halfway between distal brush 610 and proximal brush 620. In some embodiments, the intercalary brushes are set at an angle to the longitudinal axis of the head 210, so that the intersection between the brush and the surface of the side of head 210 is an ellipse. In other embodiments, at least one intercalary brush forms a spiral around the side of the head 210.

In some embodiments of a ramrod with brushes, the brushes do not comprise continuous brushing regions; a brush can comprise a plurality of brushing regions with non-brushing regions between the brushing regions. FIG. 6C shows a non-limiting example of a ramrod 200 with brushes with non-continuous brushing regions (640). In the example of FIG. 6C, each non-continuous brush forms a circle around the head 210; such a brush can also be described as a series of brushes forming an interrupted ring of brushes. The brushing regions of non-continuous brushes can form sectors of substantially circular rings, as shown in the example, sectors of substantially elliptical rings, sectors of spirals, sectors of substantially oval rings, sectors of hyperbolas, sectors of lines parallel to the longitudinal axis of the ramrod, or any combination thereof. Sectors can subtend any angle from 0 to 360°.

In some embodiments, the head can comprise patches of brush. An example of a ramrod 200 with head 210 comprising patches of brush is shown in FIG. 6D. In FIG. 6D, three patches 650, 660 of brush are shown. Two patches (650) form sectors of circles, while one (660) is substantially longitudinal to the head 210. A patch can form a sector of an ellipse or a sector of a spiral. Patches can be arranged to form circular rings, elliptical rings, spirals, lines parallel to the longitudinal axis of the ramrod, or can be arranged randomly. Sectors can subtend any angle from 0 to 360°.

The perimeter of a patch can be substantially circular, substantially elliptical, substantially oval, substantially polygonal, substantially stellate, irregular, or any combination thereof.

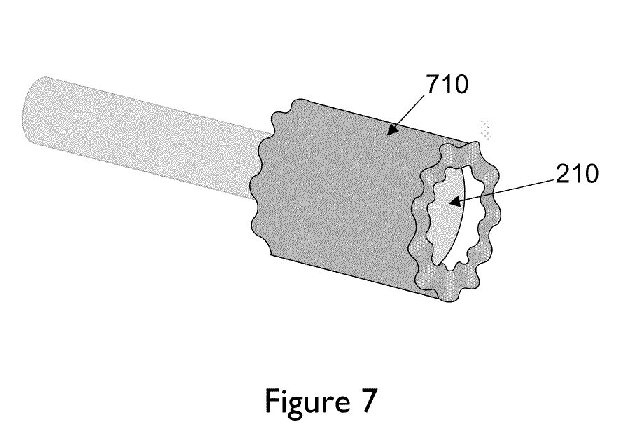

In reference to FIG. 7, in some embodiments, the head 210 of the ramrod is covered with a removable cover 710. The cover 710 fits the head 210 snugly around the sides of the head (shown cylindrical in FIG. 7) where the cover 710 contacts the bore, and the cover 710 is sufficiently snugly fitted to the top and bottom of the head 210 that the cover 710 moves substantially with the head 210 as the head traverses the bore during cleaning. In preferred embodiments, the cover is disposable. The cover can be made of flexible plastic, rubbery plastic, foam, woven material, non-woven material, chamois cloth, and any combination thereof.

The cover can be cylindrical, fitting snugly to the head of the ramrod, or it can be a flat sheet wrapped snugly around the head of the ramrod. A cylindrical cover can comprise openings to make it easier to mount the cover on the ramrod head. The cover can comprise a gathered or elasticized top, as shown or can comprise a top of substantially the same shape as the distal end of the head, at least partially attached to the cylindrical side of the cover. The top can completely cover the distal end of the head or can have holes in it. It can be unitary or can be comprised of sections and can comprise fastenings.

The cover can also comprise a gathered or elasticized bottom encircling the body of the ramrod, or the bottom can be of substantially the same shape as the proximal end of the head and be at least partially attached to the cylindrical side of the cover. The bottom can be unitary or can be comprised of sections and can comprise fastenings.

The cover can comprise a single layer or a number of layers, and can also comprise at least one brush on at least one portion of its outer surface, the brush adapted to contact the surface of the bore.

The at least one brush attached to the cover 710 is firmly attached to at least one portion of the cover and with at least one portion of the brush enabled to contact at least some portion of the wall of the bore, the brush adapted to remove particulates from the walls of the bore. The brush can be positioned and shaped as described above in reference to brushes attached to the ramrod.

The brush is preferably not of metal. The brush can be comprised of rigid plastic, rubbery plastic, foam, carbon fiber, and any combination thereof. The brush can comprise wire, continuous portions, and any combination thereof.

The brush is preferably permanently attached to the cover, although it can be removably attached. The brush is preferably rigidly attached to the cover, although it can be flexibly attached thereunto.

The brush can be attached such that it is at the distal end of the head when the cover is in use, at the proximal end of the cover, or as intercalary brush in a position on the cover between the distal end of the head when the cover is in use and the proximal end of the head when the cover is in use, at a distance from the proximal end of the head when the cover is in use of 0 to approximately 100% of the length of the head.

In some embodiments, the intercalary brushes are set at an angle to the longitudinal axis of the head 210, so that, in use, the centerline of the contact between the brush and the cover is an ellipse. In other embodiments, at least one intercalary brush forms a spiral around the cover when the cover is in use.

In some embodiments of a cover with brushes, the brushes do not comprise continuous brushing regions; a brush can comprise a plurality of brushing regions with non-brushing regions between the brushing regions. The brushing regions of non-continuous brushes can form sectors of substantially circular rings, sectors of substantially elliptical rings, sectors of spirals, sectors of substantially oval rings, sectors of hyperbolas, sectors of lines parallel to the longitudinal axis of the ramrod, or any combination thereof. Sectors can subtend any angle from 0 to 360°.

In some embodiments, the cover can comprise patches of brush. A patch can form a sector of an ellipse or a sector of a spiral. Patches can be arranged to form circular rings, elliptical rings, spirals, lines parallel to the longitudinal axis of the ramrod, or can be arranged randomly. Sectors can subtend any angle from 0 to 360°.

The perimeter of a patch can be substantially circular, substantially elliptical, substantially oval, substantially polygonal, substantially stellate, irregular, or any combination thereof.

In some embodiments, the ramrod forms part of a cleaning protocol adapted to ensure that a proper standard of cleanliness is maintained for the MRI. The standard of care protocol includes at least one criterion determining when the MRI bore is to be cleaned. This criterion can be related to usage, to time, to cleanliness or any combination thereof.

Examples of usage-related criteria include cleaning the bore: before use but after a given number of uses, such as (for non-limiting example) before every use or before every third use; or after a given number of uses, such as (for non-limiting example) after every use or after every third use.

Examples of time-related criteria include cleaning the bore: after a fixed time interval has elapsed, such as every 24 hours or once a week; on a specified day, such as on a particular day of the week or a particular day of the month; and on a given date, such as (for non-limiting example) the 1^{st} of September

Examples of cleanliness-related criteria include cleaning the bore after a specified quantity of particulates are found in the bore. Determination of the quantity of particulates can be done by visual inspection; via an optical scanner counting particulates; via measuring the attenuation of signal (such as a light beam) as it travels down the walls of the bore; via measurement of the degradation of the intensity or uniformity of the magnetic field, or by any other means known in the art of measuring particulates.

When at least one of these criteria is fulfilled, the bore is cleaned by reciprocating the ramrod within the bore, either manually or via mechanical means, as described above.

What is claimed is:

1. A ramrod for cleaning the bore of an MRI device, comprising:
a. a head with cross-section substantially identical in shape to said MRI bore's cross-section, said cross section having size slightly smaller than said MRI's bore's cross-section to fit snugly and movably within said bore, said head having at least one end, said head having a longitudinal axis; and
b. a rigid extended longitudinal body rigidly connected to said at least one end, said body having a longitudinal axis;
wherein passage of said ramrod through said bore removes particulates from said bore.

2. The ramrod of claim 1, wherein said head has shape selected from a group consisting of substantially cylindrical, substantially spherical, substantially ovoid, substantially ellipsoidal, substantially conical, comprising one or more frustums, and any combination thereof.

3. The ramrod of claim 1, wherein at least one of the following is being held true (a) said longitudinal body is rigid, flexible or both, in a manner that at least one first portion is rigid and at least one second neighboring portion is flexible; (b) the material of said ramrod is selected from a group consisting of rigid plastic, hard rubbery plastic, wood, carbon fiber, a non-metallic laminate, and any combination thereof; (c) the material of the surface of said ramrod is selected from a group consisting of rigid plastic, rubbery plastic, foam, woven material, non-woven material, chamois cloth, carbon fiber and any combination thereof; (c) at least a portion of said ramrod is hollow; (d) the angle subtended between said longitudinal axis of said head and said longitudinal axis of said body is between 0 and about 5°; (e) said extended longitudinal body has cross-section selected from circular, oval, elliptical, polygonal, stellate, and any combination thereof.

4. The ramrod of claim 2, wherein at least one of the following is being held true (a) at least one pair of frustums is joined at their wide ends, at their narrow ends, narrow end to wide end, and any combination thereof; (b) at least one pair of frustums are separated by a substantially cylindrical section.

5. The ramrod of claim 1, wherein said head comprises at least one groove, said groove substantially parallel to said longitudinal axis, substantially perpendicular to said longitudinal axis, said groove disposed spirally about said head, and any combination thereof.

6. The ramrod of claim 5, wherein at least one of the following is being held true (a) said at least one groove is less than about 5 mm deep at their deepest point; (b) said at least one groove has a profile that is V-shaped, U-shaped and any combination thereof.

7. The ramrod of claim 1, wherein said extended longitudinal body comprises at least one hand grip.

8. The ramrod of claim 7, wherein at least one of the following is being held true (a) said at least one hand grip comprises at least one portion selected from a group consisting of finger grooves, a pistol grip, and any combination thereof; (b) said at least one hand grip is comprised of a member of a group consisting of rigid plastic, rubbery plastic, foam, woven material, non-woven material, chamois cloth, wood, carbon fiber and any combination thereof.

9. The ramrod of claim 1, additionally comprising at least one brush attached to at least some portion of its external surface, said brush adapted to contact at least some portion of the wall of the bore of said MRI.

10. The ramrod of claim 9, wherein at least one of the following is being held true (a) said brush is attached to said ramrod in a manner selected from rigidly or flexibly; (b) said brush is disposed about said ramrod in a manner selected from a group consisting of at least one sector of a substantially circular ring around said ramrod, at least one sector of a substantially elliptical ring around said ramrod, at least one sector of a substantially oval ring around said ramrod, at least one sector of a hyperbola around said ramrod, at least one sector of a spiral around said ramrod, a line substantially parallel to the longitudinal axis of said ramrod, a substantially circular patch on said ramrod, a substantially elliptical patch on said ramrod, a substantially oval patch on said ramrod, a substantially polygonal patch on said ramrod, a spiral around said ramrod, a patch substantially parallel to the longitudinal axis of said ramrod, randomly, and any combination thereof; (c) said at least one brush is in one of the following positions: at the distal end of the head, at the proximal end of the head, on the body at a predetermined first distance form the proximal end of the head, on the head at a predetermined second distance from the distal end of the head and any combination thereof.

11. The ramrod of claim 10, wherein said first distance is in the range of 0 to approximately 50% of the length of the body.

12. The ramrod of claim 1, additionally comprising a cover removably mountable on said head.

13. The ramrod of claim 12, wherein at least one of the following is being held true (a) said cover is disposable; (b) said cover fits said head snugly such that said cover moves substantially with said head during cleaning; (c) said cover is comprised of flexible plastic, rubbery plastic, foam, woven material, non-woven material, chamois cloth, and any combination thereof; (d) said cover is comprised of one or more layers; (e) said ramrod additionally comprising at least one brush on at least some portion of its external surface, said at least one brush adapted to contact the surface of said bore; (f) said brush is attached to said cover in a manner selected from rigidly or flexibly; (g) said at least one brush is in one of the following positions: at the distal end of the head, at the proximal end of the head, on the head at a predetermined second distance from the distal end of the head and any combination thereof.

14. The ramrod of claim 10, wherein said second distance is in the range of 0 to approximately 100% of the length of the head; further wherein said brush is disposed about said cover in a manner selected from a group consisting of at least one sector of a substantially circular ring around said ramrod, at least one sector of a substantially elliptical ring around said ramrod, at least one sector of a substantially oval ring around said ramrod, at least one sector of a hyperbola around said ramrod, at least one sector of a spiral around said ramrod, a line substantially parallel to the longitudinal axis of said ramrod, a substantially circular patch on said ramrod, a substantially elliptical patch on said ramrod, a substantially oval patch on said ramrod, a substantially polygonal patch on said ramrod, a spiral around said ramrod, a patch substantially parallel to the longitudinal axis of said ramrod, randomly, and any combination thereof.

15. The ramrod of claims 10, wherein at least one of the following is being held true (a) said brush is comprised of at least one wire, at least one continuous portion and any combination thereof; (b) said brush is comprised of rigid plastic, rubbery plastic, foam, carbon fiber, and any combination thereof; (c) said at least one sector subtends an angle of from 0 to 360°; (d) the perimeter of said patch has a shape selected from a group consisting of substantially circular, substantially elliptical, substantially oval, substantially polygonal, substantially stellate, irregular, and any combination thereof.

16. The ramrod of claim 12, wherein at least one of the following is being held true (a) said cover comprises a top shaped to fit snugly to at least some portion of the distal end of said ramrod head; (b) said cover comprises a bottom shaped to fit snugly to at least some portion of the proximal end of said ramrod head; and any combination thereof.

17. A standard of care protocol for cleaning the bore of an MRI, comprising
a. providing a ramrod with a substantially cylindrical head with cross-section substantially identical in shape to said MRI bore's cross-section, said cross section having size slightly smaller than said MRI's bore's cross-section to fit snugly and movably within said bore, said head having at least one end, said head having a longitudinal axis; and a rigid extended longitudinal body rigidly connected to said at least one end, said body having a longitudinal axis;
b. specifying at least one cleaning criterion;
c. waiting until at least one said at least one cleaning criterion is fulfilled; and
d. reciprocating said ramrod through said bore, thereby maintaining the cleanliness of said bore;
wherein by reciprocating said ramrod through said bore particulates from said bore are removed.

18. The standard of care protocol of claim 17, wherein at least one of the following is being held true (a) said cleaning criterion is selected from a group consisting of: before use of said MRI and after a predefined number of uses of said MRI; after use of said MRI, said use being the last of a predefined number of uses of said MRI; after a predefined time interval has elapsed; on a predefined day; on a predefined date; a predefined quantity of particulates are exceeded within the bore; and any combination thereof; (b) said predefined number of uses of said MRI is greater than or equal to one; (c) further comprising additional steps of examining said bore and determining the quantity of particulates therein; wherein said examination of said bore is conducted visually, mechanically, optically and any combination thereof.

19. A method of providing a homogeneous magnetic field in the Volume of interest (VOI) of an MRI device, comprising steps of:
a. providing a ramrod with a substantially cylindrical head with cross-section substantially identical in shape to said MRI bore's cross-section, said cross section having size slightly smaller than said MRI's bore's cross-section to fit snugly and movably within said bore, said head having at least one end, said head having a longitudinal axis; and a rigid extended longitudinal body rigidly connected to said at least one end, said body having a longitudinal axis; and
b. reciprocating said ramrod through said bore;
thereby removing metal debris from said bore and providing a homogeneous magnetic field in the VOI.

20. The method of cleaning the bore of claim 19, additionally comprising at least one step selected from a group consisting of (a) selecting said head shape from a group consisting of substantially cylindrical, substantially spherical, substantially ovoid, substantially ellipsoidal, substantially conical, comprising one or more frustums, and any combination thereof; (b) providing said longitudinal body to be rigid, flexible or both, in a manner that at least one first portion is rigid and at least one second neighboring portion is flexible; (c) selecting the material of said ramrod from a group consisting of rigid plastic, hard rubbery plastic, wood, carbon fiber, a non-metallic laminate, and any combination thereof; (d) selecting the material of the surface of said ramrod from a group consisting of rigid plastic, rubbery plastic, foam, woven material, non-woven material, chamois cloth, carbon fiber and any combination thereof; (e) providing said ramrod such that at least a portion of said ramrod is hollow; (f) setting the angle subtended between said longitudinal axis of said head and said longitudinal axis of said body between 0 and about 5°; (g) selecting the cross-section of said extended longitudinal body from a group consisting of circular, oval, elliptical, polygonal, stellate, and any combination thereof.

21. The method of cleaning the bore of claim 20, additionally comprising at least one step selected from a group consisting (a) comprising said head of at least one pair of frustums, said frustums joined at their wide ends, at their narrow ends, narrow end to wide end, and any combination thereof; (b) separating at least one pair of frustums by a substantially cylindrical section.

22. The method of cleaning the bore of claim 19, additionally comprising a step of providing said head comprising at least one groove, said groove substantially parallel to said longitudinal axis, substantially perpendicular to said longitudinal axis, said groove disposed spirally about said head, and any combination thereof.

23. The method of cleaning the bore of claim 22, additionally comprising at least one step selected from a group consisting (a) selecting the depth of said at least one groove to be less than about 5 mm deep at their deepest point; (b) selecting the profile of said at least one groove to be V-shaped, U-shaped and any combination thereof.

24. The method of cleaning the bore of claim 19, additionally comprising a step of providing said extended longitudinal body comprising at least one hand grip.

25. The method of cleaning the bore of claim 24, additionally comprising at least one step selected from a group consisting (a) providing said at least one hand grip comprising at least one portion selected from a group consisting of finger grooves, a pistol grip, and any combination thereof; (b) providing said at least one hand grip from a member of a group consisting of rigid plastic, rubbery plastic, foam, woven material, non-woven material, chamois cloth, wood, carbon fiber and any combination thereof.

26. The method of cleaning the bore of claim 19, additionally comprising a step of providing at least one brush attached to at least some portion of said ramrod's external surface, said brush adapted to contact at least some portion of the wall of the bore of said MRI.

27. The method of cleaning the bore of claim 26, additionally comprising at least one step selected from a group consisting (a) attaching said brush to said ramrod in a manner selected from rigidly or flexibly; (b) disposing said brush about said ramrod in a manner selected from a group consisting of at least one sector of a substantially circular ring around said ramrod, at least one sector of a substantially elliptical ring around said ramrod, at least one sector of a substantially oval ring around said ramrod, at least one sector of a hyperbola around said ramrod, at least one sector of a spiral around said ramrod, a line substantially parallel to the longitudinal axis of said ramrod, a substantially circular patch on said ramrod, a substantially elliptical patch on said ramrod, a substantially oval patch on said ramrod, a substantially polygonal patch on said ramrod, a spiral around said ramrod, a patch substantially parallel to the longitudinal axis of said ramrod, randomly, and any combination thereof; (c) positioning said at least one brush in one of the following positions: at the distal end of the head, at the proximal end of the head, on the body at a predetermined first distance form the proximal end of the head, on the head at a predetermined second distance from the distal end of the head and any combination thereof.

28. The method of cleaning the bore of claim 27, additionally comprising a step of selecting said first distance to be in the range of 0 to approximately 50% of the length of the body.

29. The method of cleaning the bore of claim 19, additionally comprising a step of providing a cover removably mountable on said head.

30. The method of cleaning the bore of claim 29, additionally comprising at least one step selected from a group consisting (a) selecting said cover to be disposable; (b) fitting said cover snugly to said head such that said cover moves substantially with said head during cleaning; (c) providing said cover of flexible plastic, rubbery plastic, foam, woven material, non-woven material, chamois cloth, and any combination thereof; (d) providing said cover of one or more layers; (e) providing at least one brush on at least some portion of said cover's external surface, said at least one brush adapted to contact the surface of said bore; (f) attaching said brush to said cover in a manner selected from rigidly or flexibly; (g) positioning said at least one brush in one of the following positions: at the distal end of the head, at the proximal end of the head, on the head at a predetermined second distance from the distal end of the head and any combination thereof; (h) disposing said brush about said cover in a manner selected from a group consisting of at least one sector of a substantially circular ring around said ramrod, at least one sector of a substantially elliptical ring around said ramrod, at least one sector of a substantially oval ring around said ramrod, at least one sector of a hyperbola around said ramrod, at least one sector of a spiral around said ramrod, a line substantially parallel to the longitudinal axis of said ramrod, a substantially circular patch on said ramrod, a substantially elliptical patch on said ramrod, a substantially oval patch on said ramrod, a substantially polygonal patch on said ramrod, a spiral around said ramrod, a patch substantially parallel to the longitudinal axis of said ramrod, randomly, and any combination thereof.

31. The method of cleaning the bore of claim 27, additionally comprising at least one step selected from a group consisting of (a) selecting said second distance to be in the range of 0 to approximately 100% of the length of the head; (b) providing said brush of at least one wire, at least one continuous portion and any combination thereof; (c) providing said brush of rigid plastic, rubbery plastic, foam, carbon fiber, and any combination thereof; (d) selecting the angle subtended by said at least one sector to be from 0 to 360°; (e) selecting the shape of the perimeter of said patch from a group consisting of substantially circular, substantially elliptical, substantially oval, substantially polygonal, substantially stellate, irregular, and any combination thereof.

32. The method of cleaning the bore of claim 29, additionally comprising at least one step selected from a group consisting of (a) providing said cover with a top shaped to fit snugly to at least some portion of the distal end of said ramrod head; (b) providing said cover with a bottom shaped to fit snugly to at least some portion of the proximal end of said ramrod head.
